(19) United States
(12) Patent Application Publication
(10) Pub. No.: US 2025/0295526 A1
(43) Pub. Date: Sep. 25, 2025

(54) SURGICAL INSTRUMENTS FOR MEMBRANE PEELING

(71) Applicant: Alcon Inc., Fribourg (CH)

(72) Inventors: Reto Grüebler, Greifensee (CH); Luca Palmerini, Basel (CH); Venjamin Stojanov, Dietlikon (CH); Christoph Siegenthaler, Eschenz (CH)

(21) Appl. No.: 19/081,206

(22) Filed: Mar. 17, 2025

Related U.S. Application Data
(60) Provisional application No. 63/567,626, filed on Mar. 20, 2024.

(51) Int. Cl. A61F 9/007

(52) U.S. Cl. CPC A61F 9/007

(57) ABSTRACT

An ophthalmic surgical instrument for peeling a retinal membrane is provided. The ophthalmic surgical instrument includes a handle, an actuator mounted to the handle, a first loop, and a second loop. The first loop extends outwardly from the handle and comprises a first plurality of protruding features configured to create an internal limiting membrane (ILM) flap in an eye. The second loop extends outwardly from the handle, is positioned within the first loop, and is configured to grasp the ILM flap. The actuator is configured to move the second loop to engage the first loop to grasp the ILM flap.

### INTRODUCTION

The internal limiting membrane (ILM) is a thin transparent membrane positioned between the vitreous and the retina of the eye. The ILM plays a role during the formation of the eye but is not required for the proper function of an adult eye. The ILM may pull at the retina and cause conditions such as macular holes, macular pucker, vitreo-macular traction syndrome, diabetic macular edema, and cystoid macular edema secondary to inflammation or venous occlusive diseases and other conditions. An epiretinal membrane (ERM) is a membrane that may form over the retina in response to damage to the retina, such as due to posterior vitreous detachment.

The ILM or ERM may need to be peeled away from the retina to prevent damage to the retina. Peeling of the ILM or ERM may also be required in preparation for surgical procedures performed on the retina. To peel the ILM or ERM, a surgical instrument is inserted through a cannula within the patient's eye globe. Forceps or a specialized scraper are typically extended from the instrument and used to raise a flap in the ILM or ERM. The flap is then grasped by the forceps and the ILM or ERM is peeled away from the retina using a circular motion. However, excess force on the forceps may result in piercing of the retina and/or other retinal damage.

It would, therefore, be an advancement in the art to reduce the risk of retinal damage resulting from ILM or ERM peeling.

### BRIEF SUMMARY

The present disclosure relates generally to ophthalmic surgical instruments for peeling a retinal membrane.

In certain embodiments, an ophthalmic surgical instrument for peeling a retinal membrane is provided. The ophthalmic surgical instrument includes a handle, an actuator mounted to the handle, a first loop extending outwardly from the handle, the first loop including a first plurality of protruding features extending from a surface of the first loop and configured to grip an internal limiting membrane (ILM) flap in an eye, and a second loop extending outwardly from the handle and positioned within the first loop, and configured to grasp the ILM flap, where the actuator is configured to move the second loop to engage the first loop to grasp the ILM flap.

In certain embodiments, another ophthalmic surgical instrument for peeling a retinal membrane is provided. The ophthalmic surgical instrument includes a handle, an actuator mounted to the handle, a first loop extending outwardly from the handle and configured to create an ILM flap in an eye, and a second loop extending outwardly from the handle and positioned within the first loop, the second loop comprising a pair of slots, where the actuator is configured to move the second loop to engage the first loop to grasp the ILM flap by traversing along the first loop as guided by the pair of slots.

In certain embodiments, another ophthalmic surgical instrument for peeling a retinal membrane is provided. The ophthalmic surgical instrument includes a handle, an actuator mounted to the handle, a first loop extending outwardly from the handle, comprising a plurality of protruding features configured to create an ILM flap in eye, and a second loop extending outwardly from the handle and positioned within the first loop, the second loop configured to grasp the ILM flap, where the actuator is configured to move the second loop to engage the first loop to grasp the ILM flap.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1A is an isometric view of a surgical instrument, according to certain embodiments.

FIG. 1B is a cutaway view of a grasping structure of the surgical instrument shown in FIG. 1A, according to certain embodiments.

FIGS. 1C-1D are isometric views of alternative grasping structures of the grasping structure shown in FIG. 1A, according to certain embodiments.

FIG. 2 is an isometric view of an alternative embodiment for actuators for controlling the grasping structure of the surgical instrument shown in FIG. 1A, according to certain embodiments.

FIG. 3A is a cross-sectional isometric view of a loop connection configuration of the surgical instrument shown in FIG. 1A, according to certain embodiments.

FIG. 3B is a cross-sectional perspective view illustrating a mechanism for actuating the loops of the grasping structure shown in FIG. 1A, according to certain embodiments.

FIG. 3C is a cross-sectional perspective view illustrating a mechanism for actuating the loops of the grasping structure shown in FIG. 1A, according to certain embodiments.

FIG. 3D is a cross-sectional perspective view illustrating a mechanism for actuating the loops of the grasping structure shown in FIG. 1A, according to certain embodiments.

FIG. 3E is a cross-sectional perspective view illustrating a mechanism for actuating the loops of the grasping structure shown in FIG. 1A, according to certain embodiments.

FIG. 3F is a cross-sectional isometric view of an alternative loop connection configuration of the surgical instrument shown in FIG. 1A, according to certain embodiments.

FIGS. 4A-4B are isometric views showing the grasping structure of FIG. 1A in various configurations, according to certain embodiments.

FIGS. 5A-5D show the peeling of an internal limiting membrane (ILM) using the grasping structure of FIG. 1A, according to certain embodiments.

FIG. 6A-6C are isometric views of alternative grasping structures, according to certain embodiments.

FIG. 7 is an isometric view of another alternative loop connection configuration, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

It will be readily understood that the components of the embodiments as generally described herein and illustrated in the appended Figures can be arranged and designed in a wide variety of different configurations. Thus, the following more detailed description of various embodiments, as represented in the Figures, is not intended to limit the scope of the present disclosure but is merely representative of various embodiments. While the various aspects of the embodiments are presented in the Figures, the Figures are not necessarily drawn to scale unless specifically indicated.

Reference throughout this specification to the term “distal” refers to a system, device, component, end, portion, or segment that is disposed closer to a patient and/or further from a console during an ophthalmic procedure; and the term “proximal” refers to the system, device, component, end, portion, or segment that is disposed further from the patient and/or closer to the console during the ophthalmic procedure.

Reference throughout this specification to the term “membrane” refers to a retinal membrane in an eye such as, for example, an internal limiting membrane (ILM) or an epiretinal membrane (ERM); and the term “flap” refers to a flap created in the membrane such as, for example, an ILM flap or an ERM flap.

Retinal membrane peeling is a common surgical technique used in ophthalmic procedures during treatment of conditions such as macular holes, macular pucker, vitreo-macular traction syndrome, diabetic macular edema, and cystoid macular edema secondary to inflammation or venous occlusive diseases and other conditions. Current retinal membrane peeling techniques employ the use of surgical instruments such as forceps, scrapers, and/or pics. Such instruments may be used to create a tear in the retinal membrane, which is then grasped and peeled and/or removed.

However, a surgeon using such instruments may have difficulty determining a distance between the instrument and the retinal membrane (e.g., a transparent 3 micrometer (μm) thick membrane), which can result in the surgeon accidentally scraping and tearing the retinal membrane, and/or penetrating too far into the retinal membrane. This can result in unintentional and potentially permanent damage to the retina, disturbing its function and/or causing various other retinal-related complications. Further, when a conventional scraper is used to create a tear in the retinal membrane, an additional instrument is needed to grasp and peel the membrane, which increases other surgical risks associated with using multiple instruments and/or associated with frequent entry/exit of the eye. As such, current retinal membrane peeling instruments present a variety of limitations.

Accordingly, the surgical instruments described herein overcome many of the limitations associated with current instruments used for peeling the retinal membrane.

Certain embodiments described herein provide improved surgical instruments for use in ophthalmic procedures. More particularly, certain embodiments provide surgical instruments that safely and more efficiently create tears in, and peel, the retinal membrane. Such surgical instruments employ the use of grasping structures with multiple loops that can perform both retinal membrane tearing and peeling with limited applied force, which reduces unintentional retinal damage and retinal shredding-related risks.

Certain embodiments of the present disclosure are directed to ophthalmic surgical instrument for peeling a retinal membrane. The ophthalmic surgical instrument includes a handle, an actuator mounted to the handle, a first loop, and a second loop. The first loop includes a first plurality of protruding features extending from a surface of the first loop and configured to grip an ILM flap in an eye. The second loop extends outwardly from the handle, is positioned within the first loop, and is configured to grasp the ILM flap. The actuator is configured to move the second loop to engage the first loop to grasp the ILM flap.

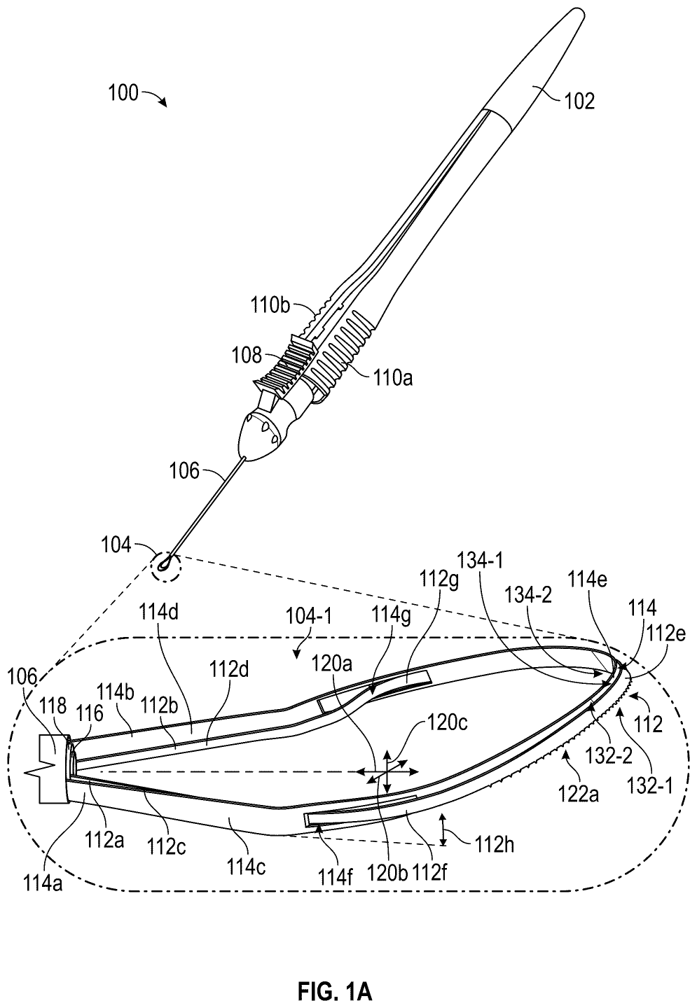

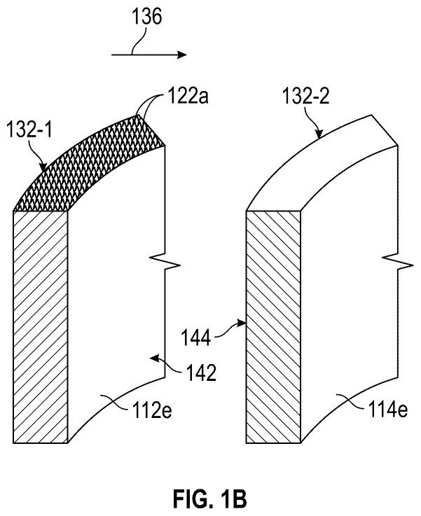

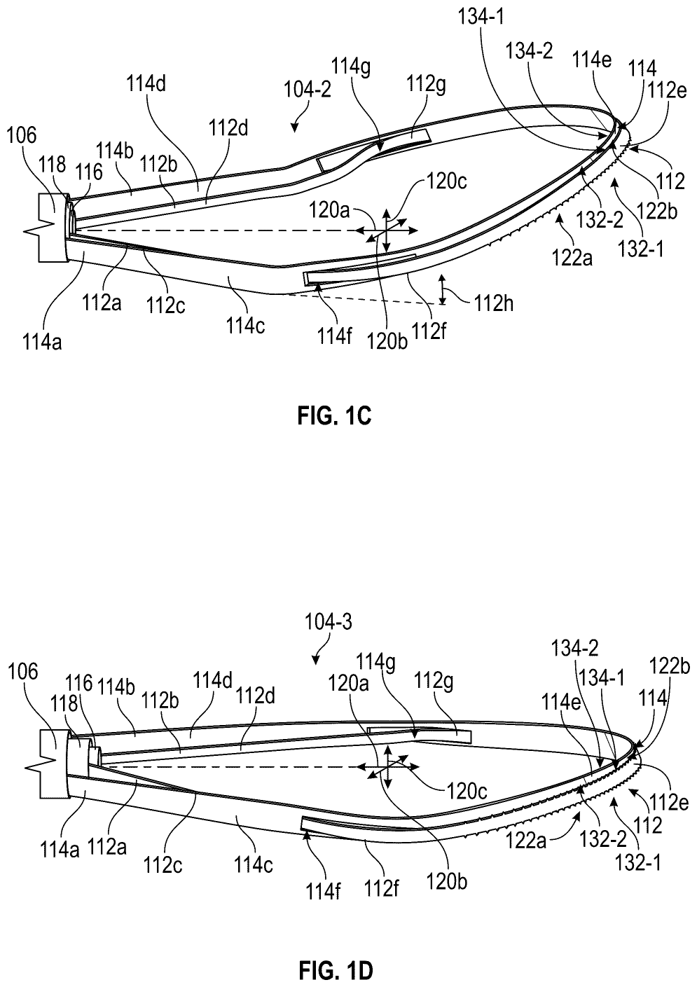

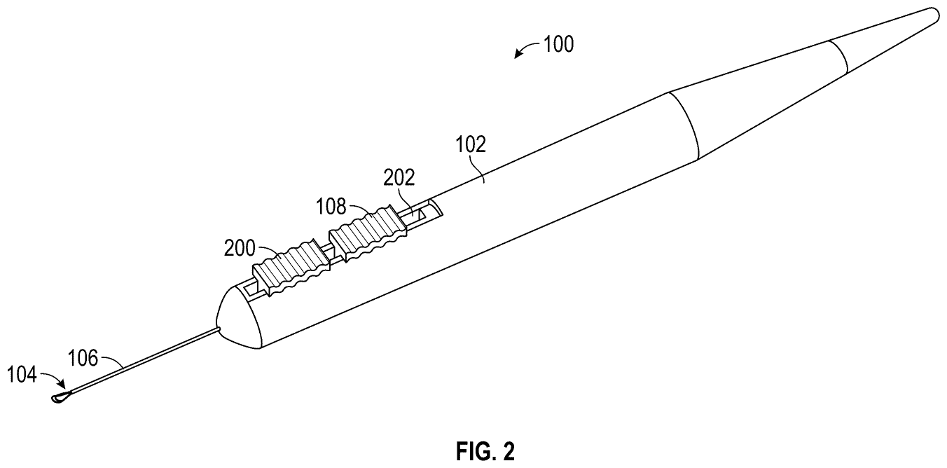

FIG. 1A is an isometric view of a surgical instrument 100, according to certain embodiments. The surgical instrument 100 includes a handle 102, a grasping structure 104-1, an outer tube 106, a slider 108, and clamshell arms 110a and 110b. The handle 102 is sized and contoured to be grasped by a hand of a surgeon performing an ophthalmic surgical procedure, such as peeling of a membrane from a retina of a patient's eye, including an ILM or an ERM. The grasping structure 104-1 is extendable from a distal end of the outer tube 106, which further includes a proximal end connected to the handle 102. The slider 108 is a manual control structure (e.g., an actuator) mounted to the handle 102. However, the handle 102 may have one or more manual control structures (e.g., actuation mechanisms) disposed thereon. The manual control structures shown are only exemplary, and other manual control structures may also be used, such as a deformable basket or a second slider as shown in FIG. 2.

In FIG. 1A, the grasping structure 104-1 includes a first loop 112 and a second loop 114. The first loop 112 may be an outer loop (e.g., distal to the second loop 114) and may be referred to as an “outer grasping member” of the grasping structure 104-1. The second loop 114 may be an inner loop (e.g., proximal to the first loop 112) and may be referred to as an “inner grasping member” of the grasping structure 104-1. In some embodiments, the outer tube 106 and/or the second loop 114 are translatable relative to the first loop 112. For example, one of the slider 108 and the clamshell arms 110a, 110b is coupled to the outer tube 106, and the other of the slider 108 and clamshell arms 110a, 110b is coupled to the second loop 114 via the second inner tube 118.

In use, the outer tube 106 may be extended over the first loop 112 and second loop 114, such as while the outer tube 106 is inserted into or withdrawn from a cannula (e.g., referred to as a trocar cannula) inserted in the patient's eye. The outer tube 106 may then be withdrawn or retracted by the slider 108, thereby extending the first loop 112 and second loop 114 relative to the outer tube 106. And as discussed in greater detail below, the second loop 114 may then be translated toward the first loop 112 in order to grasp the membrane between the first loop 112 and the second loop 114.

The first loop 112 and second loop 114 may be made of a highly flexible material, such as nitinol (a nickel titanium alloy), spring steel, or other material. The high flexibility enables the first loop 112 and second loop 114 to elastically deform in order to fit within the outer tube 106 and, when extended from the outer tube 106, expand to a size that is much wider than an outer diameter of the outer tube 106, such as at least two times, four times, eight times, or at least 16 times the outer dimeter of the outer tube 106.

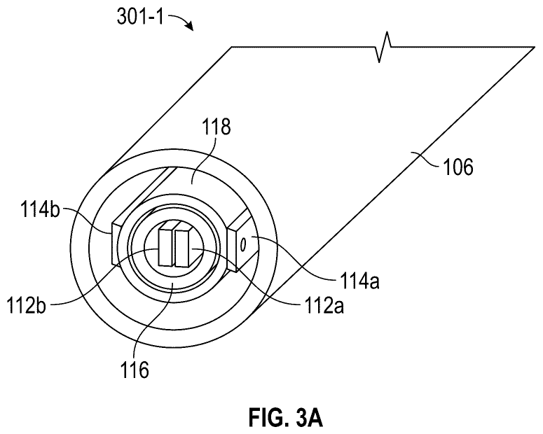

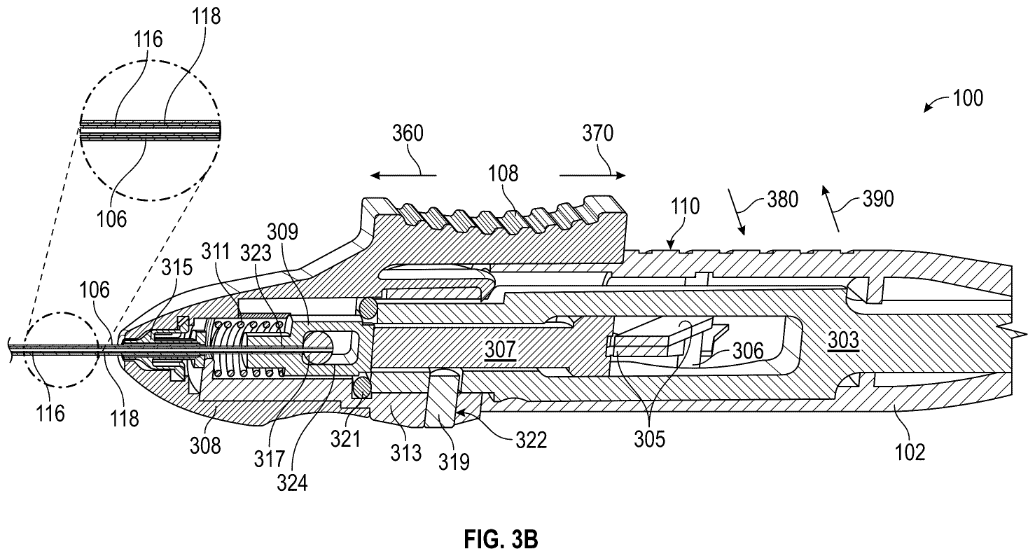

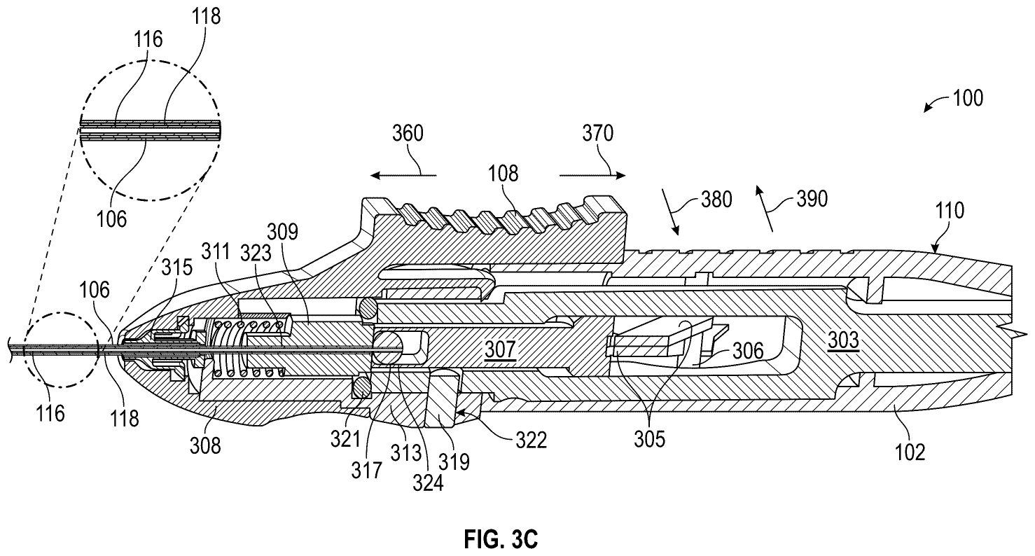

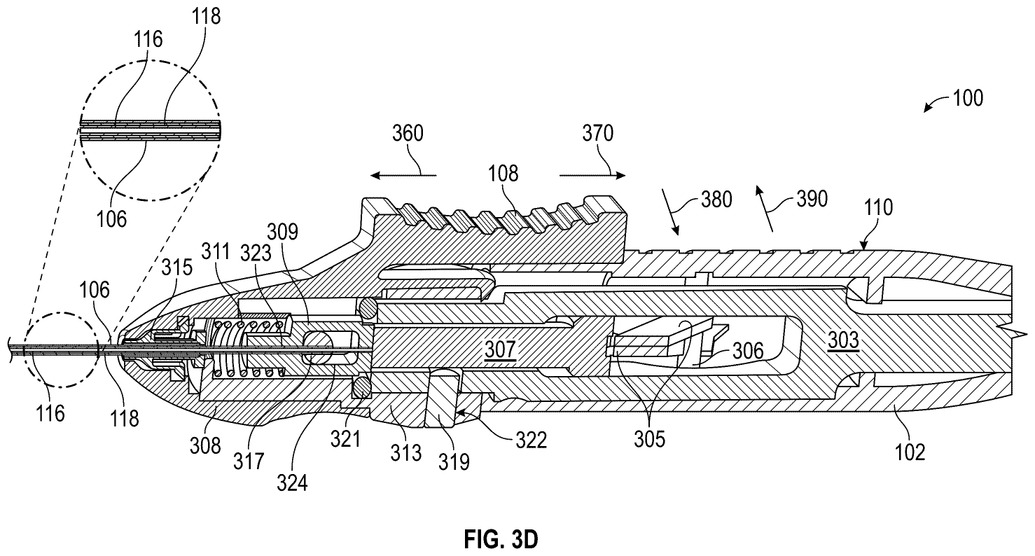

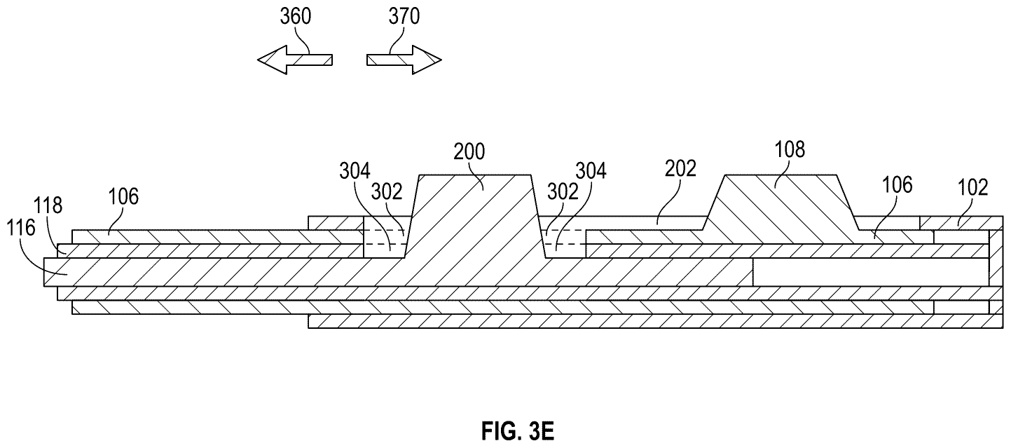

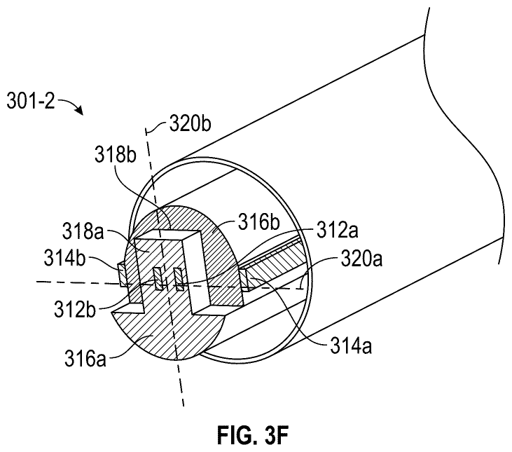

In certain embodiments, the first loop 112 has ends 112a, 112b fastened to a first inner tube 116 disposed within a second inner tube 118. In certain embodiments, the second loop 114 has ends 114a, 114b fastened to the second inner tube 118, which is slidably positioned within the outer tube 106. The outer tube 106, the first inner tube 116, and the second inner tube 118 may be made of nitinol, stainless steel, spring steel, rigid polymer, or other material. The loop connection configuration of the first loop 112, the second loop 114, the outer tube 106, the first inner tube 116, and the second inner tube 118 is described in further detail with reference to FIG. 3A.

The outer tube 106 defines a longitudinal direction 120a parallel to and collinear with an axis of symmetry of the outer tube 106. The axes of symmetry of the first inner tube 116 and second inner tube 118 are substantially (e.g., within 0.5 millimeters (mm)) collinear with the longitudinal direction 120a and substantially (e.g., within 5 degrees of) parallel to the longitudinal direction 120a. A transverse direction 120b may also be defined as perpendicular to the longitudinal direction 120a such that the ends 112a, 112b of the first loop 112 are offset from one another along the transverse direction 120b and the ends 114a, 114b of the second loop 114 are offset from one another along the transverse direction 120b. A vertical direction 120c may be defined as perpendicular to the longitudinal direction 120a and the transverse direction 120b.

In certain embodiments, the first loop 112 may include straight portions 112c, 112d extending from the ends 112a, 112b, respectively. The straight portions 112c, 112d may be intersected by a plane containing the longitudinal direction 120a and transverse direction 120b (“the longitudinal-transverse plane”). The straight portions 112c, 112d may diverge from one another in the longitudinal transverse plane, i.e., flare outwardly from one another with distance from the distal end of the outer tube 106. As used herein, “straight” may be understood as having a radius of curvature in the longitudinal-transverse plane of greater than 1 centimeter (cm). As used herein, the longitudinal transverse plane includes the plane containing both the longitudinal direction 120a and transverse direction 120b.

The straight portions 112c, 112d are connected to one another by a rounded end portion 112e. The rounded end portion 112e may be either (a) formed to hold a rounded shape absent an external force or (b) the result of bending of the first loop 112 and securement of the ends 112a, 112b to the first inner tube 116. The rounded shape may be circular, elliptical, or any arbitrary rounded shape.

Rounded end portion 112e may further be secured to the straight portions 112c, 112d by guiding portions 112f, 112g. The guiding portions 112f, 112g may function as guides for the second loop 114. For example, when the second loop 114 is traversed along the first loop 112 as described in further detail below, the guiding portions 112f, 112g facilitate proper and linear movement of the second loop 114 along the first loop 112. In other embodiments, there are no discrete guiding portions. In such embodiments, some or all of the expanse between the rounded end portion 112e and the ends 112a, 112b provides guidance for the second loop 114.

The guiding portions 112f, 112g may have the same height or a reduced height (e.g., perpendicular to the longitudinal-transverse plane) and/or thickness (e.g., parallel to the longitudinal-transverse plane) relative to one or both of the rounded end portion 112e and the straight portions 112c, 112d. In some embodiments, the height and thickness of the guiding portions 112f, 112g is substantially (e.g., within 10 percent of) the same as that of the straight portions 112c, 112d and the rounded end portion 112e. In some embodiments, the height of the guiding portions 112f, 112g may be between 0.25 and 0.75 times or between 0.4 and 0.6 times the height of the straight portions 112c, 112d and the rounded end portion 112e.

In the illustrated embodiments, the rounded end portion 112e includes a first plurality of protruding features 122a configured to create an ILM flap. That is, the first protruding features 122a are configured to penetrate the ILM when pressed onto the ILM, thereby facilitating the creation of the ILM flap when moved along the ILM surface by a surgeon. The first protruding features 122a may include a barbed or tooth-like morphology (best seen in FIG. 1B), or other similar shaped morphology configured to penetrate the ILM. For example, in certain embodiments, one or more of the first plurality of protruding features 122a may be triangular, tetrahedral, pyramidal, cuboid, cylindrical, capsular, hemispherical, or similar in shape. The protruding features 122a protrude transversely from the rounded end portion 112e in a direction that is substantially parallel to the vertical direction 120c.

In certain embodiments, one or more of the first plurality of protruding features 122a protrude from the rounded end portion 112e by a length that is substantially (e.g., within 10 percent of) the same as a thickness of the retinal membrane. For example, the length of the first protruding features 122a outward from the lower surface 132-1 is between 0.8 and 8 microns (e.g., 1 and 7 microns, 2 and 6 microns, or 3 and 5 microns). The first protruding features 122a may be arranged along a length of the rounded end portion 112e and may stop at or before the guiding portions 112f, 112g. In certain embodiments, there may be at least two protruding features (e.g., at least 10, 20, 30, 40, or 50 protruding features). In still further embodiments, there is only one protruding feature 122a.

It is desirable that a lower surface 132-1 of the rounded end portion 112e be relatively parallel to the retina to reduce risk of puncture. In response to pressure exerted on the rounded end portion 112e by the membrane during use, the rounded end portion 112e will rotate (or bend) until the lower surface 132-1 of the rounded end portion 112e is resting on the membrane, thereby increasing the surface area in contact with the membrane and reducing risk of puncturing too deep. The cross-sectional shape of the rounded end portion 112e may have a height (e.g., perpendicular to the longitudinal-transverse plane) that is similar to the thickness (e.g., parallel to the longitudinal-transverse plane) such that the rounded end portion 112e substantially flexes in a plane parallel to the longitudinal direction 120a and the vertical direction 120c (“the longitudinal-vertical plane”), such as substantially (e.g., within 10 percent of) the same as the thickness. This may facilitate the lower surface 132-1 of the rounded end portion 112e providing a broad surface that resists penetrating the retina.

In some implementations, parts of the guiding portions 112f, 112g may further define a bend in the absence of any deforming force such that the rounded end portion 112e defines an angle 112h relative to the longitudinal-transverse plane and is raised above the longitudinal-transverse plane. The angle 112h may further encourage the rounded end portion 112e to rotate when pushed against a membrane rather than possibly puncturing the membrane and retina. The rounded end portion 114e may also be disposed at the same angle 112h or different angle relative to the longitudinal-transverse plane. Flexibility of the first loop 112 and second loop 114 enable the first loop 112 and second loop 114 to nest regardless of size and angle when undeformed.

In certain embodiments, the second loop 114 may include straight portions 114c, 114d extending from the ends 114a, 114b, respectively. The straight portions 114c, 114d may be intersected by the longitudinal-transverse plane. The straight portions 114c, 114d may diverge from one another in the longitudinal transverse plane, i.e., flare outwardly from one another with distance from the distal end of the outer tube 106.

The straight portions 114c, 114d may be connected to one another by a rounded end portion 114e. The rounded end portion 114e may be either (a) formed to hold a rounded shape absent an external force or (b) the result of bending of the second loop 114 and securement of the ends 114a, 114b to the second inner tube 118. The rounded end portion 114e may be angled relative to the longitudinal-transverse plane by the same angle as the rounded portion 112e of the first loop 112 or parallel relative to the longitudinal-transverse plane.

Absent a deforming force, the rounded end portion 114e may have an outer surface having a size in a plane of curvature (e.g., the longitudinal-transverse plane or a plane oriented at the angle relative to the longitudinal-transverse plane) that is substantially equal to a size of the outer surface of the rounded end portion 112e, a size of the inner surface of the rounded end portion 112e, or smaller than the inner diameter of the rounded end portion 112e. Flexibility of the first loop 112 and the second loop 114 enable the first loop 112 and second loop 114 to nest regardless of size when undeformed.

The straight portions 114c, 114d may have an increased height and/or thickness relative to the straight portions 112c, 112d and/or the rounded end portion 114e such that the second loop 114 is less flexible than the first loop 112 and such that a pair of slots 114f, 114g are formed between the straight portions 114c, 114d and the rounded end portion 114e and are disposed over the guiding portions 112f, 112g of the first loop 112. In other words, the first loop 112 is disposed through the slots 114f, 114g. Rounded end portion 114e may be secured to the straight portions 114c, 114d by slots 114f, 114g.

The slots 114f, 114g enable the second loop 114 to slide along guiding portions 112f, 112g of the first loop 112. For example, when the second loop 114 is traversed along the first loop 112 to engage with the first loop 112 as described in further detail below, the slots 114f, 114g along with guiding portions 112f, 112g of the first loop 112 facilitate proper movement of the second loop 114 along the first loop 112.

The slots 114f, 114g may define an opening having the same height or an increased height (e.g., perpendicular to the longitudinal-transverse plane) and/or thickness (e.g., parallel to the longitudinal-transverse plane) relative to one or more of the guiding portions 112f, 112g, the straight portions 112c, 112d, and the rounded end portion 112e. In some embodiments, the height of the slots 114f, 114g is substantially (e.g., within 10 percent of) the same as that of the guiding portions 112f, 112g. In some embodiments, the height of the slots 114f, 114g may be between 1.25 and 1.75 times or between 1.4 and 1.6 times the height of the guiding portions 112f, 112g. The opening defined by the slots 114f, 114g may have a width that is long enough to accommodate a widest point (e.g., in the transverse direction 120b) of the first loop 112.

Although the slots 114f, 114g are shown as having a rectangular shaped opening, the slots 114f, 114g may also have a circular, cylindrical or other similar shaped opening.

As discussed in greater detail below, the first loop 112 may be used to create a flap in the membrane, which may require a degree of pressure to be exerted on the membrane. In contrast, the second loop 114 need only press the flap against the first loop 112. Accordingly, the second loop 114 may be made less flexible in order to sufficiently press the flap against the first loop 112.

FIG. 1B is a cutaway view of the grasping structure 104-1 of the surgical instrument 100 shown in FIG. 1A, according to certain embodiments. In the embodiments of FIG. 1B, one or both of the lower surfaces 132-1 and 132-2 of the rounded end portion 112e and rounded end portion 114e, respectively, may have first protruding features 122a formed thereon to facilitate gripping of the membrane. For example, the lower surfaces 132-1, 132-2 may have first protruding features 122a, such as barbs, formed thereon. For the rounded end portion 112e, the first protruding features 122a may point toward the rounded end portion 114e, as indicated by arrow 136. Stated differently, the first protruding features 122a on the rounded end portion 114e can be oriented such that movement of the rounded end portion 112e relative to the membrane will be resisted more for relative movement of the rounded end portion 112e toward the rounded end portion 114e than for relative movement away from the rounded end portion 114e. In this manner, the first protruding features 122a enhance the ability of the rounded end portion 112e to pull the membrane and raise a flap between the rounded end portions 112e, 114e. One or more of the first plurality of protruding features 122a may be tapered such that the resistance of the membrane to penetration by the first protruding features 122a increases with depth. This reduces the risk of the first protruding features 122a passing completely through the membrane. In some applications, a tapered shape for the first protruding features 122a prevents the lower surface 132-1 of the rounded end portion 112e from actually contacting the membrane during use.

In the embodiments of FIG. 1B, the rounded end portion 114e lacks protruding features on the lower surface 132-2. In other embodiments, protruding features 122 are included only on an outer surface 144 of the rounded end portion 114e. In such embodiments, one or more of the first protruding features 122a may point in the opposite direction from the protruding features on the rounded end portion 112e such that the first protruding features 122a improve the ability of the rounded end portion 114e to push the membrane toward the rounded end portion 112e. Again, in other embodiments, no protruding features 122 are formed on the lower surface 132-2 of the rounded end portion 114e such that the rounded end portion 114e is primarily or exclusively responsible for grasping the flap.

In some embodiments, an inner surface 142 of the rounded end portion 112e (surface facing the rounded end portion 114e) and an outer surface 144 of the rounded end portion 114e (surface facing the rounded end portion 112e) are textured, barbed, coated with a gripping material (e.g., silicone) in order to resist slipping of the flap when grasped between the rounded end portion 112e and rounded end portion 114e.

FIGS. 1C-1D are isometric views of alternative grasping structures of the grasping structure shown in FIG. 1A, according to certain embodiments. Turning to FIG. 1C, an isometric view of an alternative grasping structure 104-2 is shown. The grasping structure 104-2 may replace the grasping structure 104-1 of the surgical instrument 100. In certain embodiments, the grasping structure 104-2 includes the outer tube 106, the first loop 112, and the second loop 114 as described with reference to FIG. 1A. That is, the first loop 112 includes ends 112a, 112b, straight portions 112c, 112d, guiding portions 112f, 112g, a rounded end portion 112e, and the first plurality of protruding features 122a, and the second loop 114 includes ends 114a, 114b, straight portions 114c, 114d, and a rounded end portion 114e, where the rounded end portions 112e, 114e define angle 112h relative to the longitudinal-transverse plane as described with reference to FIG. 1A.

However, as opposed to the grasping structure 104-1, the second loop 114 of the grasping structure 104-2 comprises a second plurality of protruding features 122b protruding from the lower surface 132-2 of the rounded end 114e, in addition to the first plurality of protruding features 122a protruding from the lower surface 132-1 of the rounded end 112e. The second protruding features 122b are similar to the first protruding features 122a as described with reference to FIGS. 1A-1B. That is, the second protruding features 122b can be configured to help create the ILM flap by penetrating the ILM when pressed onto the ILM, thereby facilitating the creation of the ILM flap when moved along the ILM surface by a surgeon. One or more of the second protruding features 122b may include a barbed or tooth-like morphology (similar to the first plurality of protruding features 122a seen in FIG. 1B), or other similar shaped morphology configured to penetrate the ILM, as described above. Generally, the second protruding features 122b protrude transversely from the lower surface 132-2 (e.g., first side or bottom side) of the rounded end portion 114e in a direction that is substantially parallel to the vertical direction 120c.

In certain embodiments, one or more of the second protruding features 122b protrude from the rounded end portion 114e by a length that is substantially (e.g., within 10 percent of) the same as a thickness of the retinal membrane. For example, the length of the second protruding features 122b outward from the lower surface 132-1 may be between 0.8 and 8 microns (e.g., 1 and 7 microns, 2 and 6 microns, or 3 and 5 microns). The second protruding features 122b may be arranged along a length of the rounded end portion 114e, and may stop at or before the slots 114f, 114g. In certain embodiments, there are at least two protruding features 122b (e.g., at least 10, 20, 30, 40, or 50 protruding features). In still further embodiments, there is only one protruding feature 122b.

FIG. 1D is an isometric view of another alternative grasping structure 104-3, according to certain embodiments. The grasping structure 104-3 may replace the grasping structure 104-1 of the surgical instrument 100. In certain embodiments, the grasping structure 104-3 includes the outer tube 106, the first loop 112, and the second loop 114 as described with reference to FIG. 1C. That is, the first loop 112 includes ends 112a, 112b, straight portions 112c, 112d, guiding portions 112f, 112g, a rounded end portion 112e, and the first plurality of protruding features 122a, and the second loop 114 includes ends 114a, 114b, straight portions 114c, 114d, and a rounded end portion 114e as described with reference to FIG. 1C.

However, as opposed to the grasping structure 104-2, the first loop 112 and the second loop 114 do not define an angle 112h, and the second protruding features 122b are arranged on the upper surface 134-1 of the rounded end 112e of the first loop 112 instead of the lower surface 142-2 of the rounded end 114e of the second loop 114. In certain embodiments, the rounded ends 112e, 114e being disposed along the same plane as the straight portions 112c, 112d, and 114c, 114d is a result of no angle 112h. Flexibility of the first loop 112 and second loop 114 enable the first loop 112 and second loop 114 to nest and rotate when pushed against a membrane, regardless of the lack of angle 112h.

Generally, the second protruding features 122b may be arranged along the upper surface 134-1 in a similar manner as described with reference to the lower surface 142-2 in FIG. 1C. For example, one or more of the second protruding features 122b may protrude transversely from the upper surface 134-1 (e.g., second side or top side) of the rounded end portion 112e in a direction that is substantially parallel to the vertical direction 120c. In certain embodiments, one or more of the second protruding features 122b protrude from the rounded end portion 112e by a length that is substantially (e.g., within 10 percent of) the same as a thickness of the retinal membrane. For example, the length of the second protruding features 122b may be between 0.8 and 8 microns (e.g., 1 and 7 microns, 2 and 6 microns, or 3 and 5 microns). The second protruding features 122b may be arranged along a length of the rounded end portion 112e and may stop at or before the guiding portions 112f, 112g. In certain embodiments, there are at least two protruding features 122b (e.g., at least 10, 20, 30, 40, or 50 protruding features). In still further embodiments, there is only one protruding feature 122b.

Note that any features of the grasping structures 104-1, 104-2, and/or 104-3 may be implemented separately or in any combination on a grasping structure of the surgical instrument 100. As an example, a grasping structure 104 as described herein may include a plurality of protruding features on the lower surface 132-1 and/or upper surface 134-1 of the first loop 112, and/or on the lower surface 132-2 and/or upper surface 134-2 of the second loop 114.

FIG. 2 is an isometric view of an alternative embodiment for actuators for controlling the grasping structure 104 of the surgical instrument 100 shown in FIG. 1A, according to certain embodiments. Referring to FIG. 2, various actuation mechanisms may be used to manually control translation of the outer tube 106 and the second inner tube 118. In some embodiments, the clamshell arms 110a, 110b may be replaced with a second slider 200 that is slidably mounted to the handle 102. In the illustrated embodiment, the sliders 108, 200 both slide within a common slot 202 defined by the handle 102. Accordingly, one slider 108 may control actuation of the outer tube 106, and the other slider 200 may control actuation of the second loop 114, or vice versa.

FIG. 3A is a cross-sectional isometric view of a loop connection configuration 301-1 of the surgical instrument 100 shown in FIG. 1A, according to certain embodiments. The loop connection configuration 301-1 provides high variability in loop orientation and low external loop connection robustness.

At the proximal end of the grasping structure 104, the ends 112a, 112b of the first loop 112, the ends 114a, 114b of the second loop, the first inner tube 116, and the second inner tube 118 are disposed within the outer tube 106. The ends 112a, 112b of the first loop 112 are centrally disposed within an inner circumference of the first inner tube 116. The ends 114a, 114b of the second loop 114 are disposed along an outer circumference of the second inner tube 118, for example, at opposite sides of the second inner tube 118. The outer circumference of the second inner tube 118 is within an inner circumference of the outer tube 106, and the outer circumference of the first inner tube 116 is within an inner circumference of the second inner tube 118.

In certain embodiments, a gap exists between the ends 112a, 112b and the inner circumference of the first inner tube 116, and between the ends 114a, 114b and the inner circumference of the outer tube 106. The ends 112a, 112b and the ends 114a, 114b may be attached to the first inner tube 116 and the second inner tube 118, respectively, by welding, an adhesive (e.g., glue), a pin, a screw, or other similar attaching feature.

In certain embodiments, the outer tube 106, the first inner tube 116, and/or the second inner tube 118 may be moved along the longitudinal plane 120a via manipulation of an actuator (e.g., the slider 108 and the clamshell arms 110a, 110b) by a user. For example, in certain embodiments, as described with further reference to FIGS. 3B and 3C, the second inner tube 118 is moved along the longitudinal plane 120 via actuation by the clamshell arms 110a, 110b, and the outer tube 106 is moved along the longitudinal plane 120 via actuation by the slider 108, while the first inner tube 116 is stationary. In certain embodiments, as described with further reference to FIG. 3D, the first inner tube 116 is moved along the longitudinal plane 120 via actuation by the clamshell arms 110a, 110b, and the outer tube 106 is moved along the longitudinal plane 120 via actuation by the slider 108, while the second inner tube 118 is stationary.

Turning now to FIG. 3B, a partial cross-sectional perspective view of an exemplary mechanism for actuating the loops 112 and 114 of the surgical instrument 100 is shown. Because FIG. 3B is shown as a cross-sectional view, some or all of the components shown in FIG. 3B may be replicated across a longitudinal plane for components of the other half of the surgical instrument 100. Further, the illustrated mechanism is only exemplary, and the actual size and relative position of components may vary. For purposes of FIG. 3B, “first actuator” refers to the slider 108 and “second actuator” refers to clamshell arms 110a, 110b.

In FIG. 3B, the clamshell arms 110a, 110b (clamshell arm 110b is shown in FIG. 3B) are operably coupled to the second inner tube 118 (attached to the second loop 114) to actuate the second loop 114, and the slider 108 is operably coupled to the outer tube 106 disposed over the second inner tube 118 to actuate the outer tube 106.

As shown, the surgical instrument 100 includes the handle 102, which comprises the clam shell arms 110a, 110b. A stationary frame 303 is fixedly disposed within the handle 102 and supports various internal components of the surgical instrument 100 as described herein. Additionally, the stationary frame 303 is configured to couple the handle 102 with an adapter 313 at a distal end of the handle 102. In certain embodiments, the adapter 313 is removably coupled to the stationary frame 303 via a locking pin 319, which fixedly secures the adapter 313 to the stationary frame 303, and thus the handle 102, when locked in place within an opening 322 of the stationary frame 303. Disposed around a distal end of the adapter 313 is a slider head 308. The slider head 308 couples to the slider 108 and is configured to translate along, or slide along, the longitudinal plane 120a and the adapter 313 upon actuation of the slider 108 by the user. Accordingly, the slider head 308 is movably coupled to the adapter 313.

In certain embodiments, an elastomeric ring 321 (e.g., an O-ring) may be disposed between the slider head 308 and/or the adapter 313 and the stationary frame 303 to facilitate ease of assembly between the slider head 308 and/or the adapter 313 and the handle 102.

In certain embodiments, the slider head 308 is attached to the outer tube 106. Accordingly, actuation of the slider 108, and thus, the slider head 308, causes translation of the outer tube 106. In the embodiments of FIG. 3B, the slider head 308 is indirectly coupled to the outer tube 106 via a connector 315 disposed within the slider head 308. In other embodiments, however, the slider head 308 is directly attached to the outer tube 106 (without the connector 315). During use, the slider 108 may be actuated in a first direction 360 to translate the outer tube 106 outwardly from the surgical instrument 100 and over the first loop 112 and the second loop 114, to sheathe/compact the first loop 112 and the second loop 114 during, e.g., insertion/retraction of the surgical instrument 100 into/out of a cannula and/or the patient's eye. Similarly, the slider 108 may be actuated in a second direction 370, opposite the first direction 360, to translate the outer tube 106 toward the surgical instrument 100 and unsheathe the first loop 112 and the second loop 114, and/or the first inner tube 116 and the second inner tube 118, from a distal end of the outer tube 106 to enable membrane peeling.

Each clamshell arm 110a, 110b is operably coupled to one of two lever arms 305 via a corresponding pin joint 306. The lever arms 305, in turn, engage with a piston 307, which is slidably disposed within and supported by the stationary frame 303. Upon compression of the clamshell arms 110a, 110b (e.g., inwards) by a user of the surgical instrument 100, the lever arms 305 will rotate about the pin joints 306 and push distally against the piston 307, causing the piston 307 to slide distally along the longitudinal plane 120a. In certain embodiments, the rotation of the lever arms 305 to push against the piston 307 is facilitated, in part, by the lever arms 305 banking and sliding distally against sidewalls of the handle 102 upon compression of the clamshell arms 110a, 110b.

The piston 307 engages with a sliding base 309 disposed within the adapter 313. The sliding base 309 is configured to translate, or slide, along the longitudinal plane 120a within the adapter 313. The sliding base 309 engages with a spring 311 seated at a proximal portion of the adapter 313. The spring 311 is configured to bias the sliding base 309 proximally, toward the handle 102 and against the piston 307. In certain embodiments, the spring 311 is configured to be disposed around at least a portion of a proximal end of the sliding base 309, as shown in FIG. 3B, and engage with a ledge or other holding surface of the sliding base 309, to facilitate alignment and engagement of the spring 311 and the sliding base 309. Note that other types of biasing devices, other than the spring 311, are also contemplated for use with the sliding base 309.

The sliding base 309 has a conduit 323 formed therein that axially extends through at least a portion of the sliding base 309 from the proximal end of the sliding base 309. As shown, each of the first inner tube 116 and the second inner tube 118 extends through at least a portion of the conduit 323. In the embodiments of FIG. 3B, the second inner tube 118 is fixedly attached to the sliding base 309 within the conduit 323. Accordingly, in such embodiments, translation of the sliding base 309 within the adapter 313 causes actuation of the second inner tube 118, and thus, actuation of the second loop 114.

In certain embodiments, the sliding base 309 has a cavity 324 formed therein. A pin 317, which may be a welding pin, is fixedly attached to the adapter 313 and extends into the cavity 324. In the embodiments of FIG. 3B, the first inner tube 116 extends into the cavity 324 and fixedly attaches to the pin 317. Accordingly, in such embodiments, the first inner tube 116, and thus, the first loop 112, may be stationary. In such embodiments, the sliding base 309 is able to translate distally and proximally without being impeded by the pin 317 as a result of the cavity 324 formed therein.

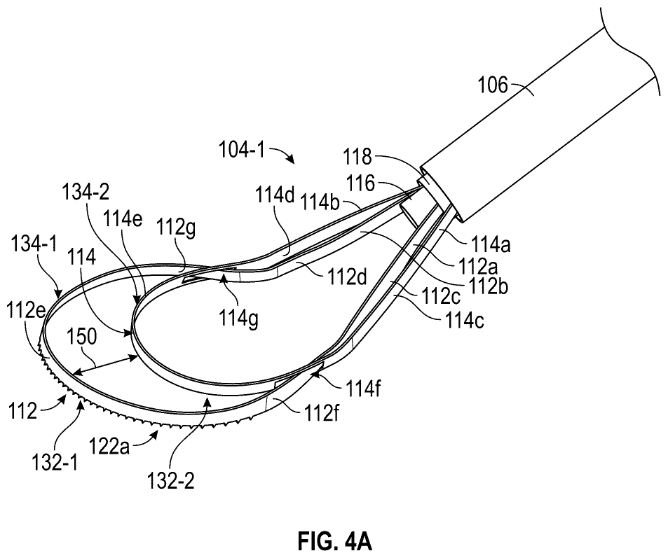

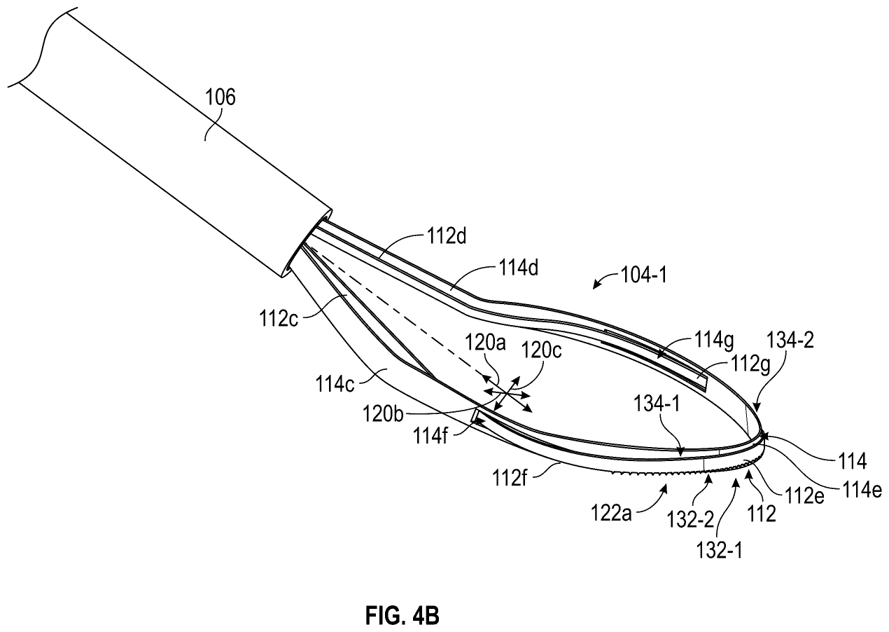

In an “open” configuration of the surgical instrument 100 in FIG. 3B (described further with reference to FIG. 4A), the clamshell arms 110a, 110b are disposed in a first, uncompressed position (or initial position). In this configuration, the first loop 112 and second loop 114 are open, meaning that the rounded end portions 112e and 114e of the first loop 112 and second loop 114, respectively, do not contact each other. To arrive at a “closed” configuration of the surgical instrument 100 for grasping a membrane flap (described further with reference to FIG. 4B), the clamshell arms 110a, 110b are compressed (e.g., actuated in a first direction 380) by the user to a second, compressed position. When the clamshell arms 110a, 110b are compressed, the lever arms 305 push distally against the piston 307, which in turn presses the sliding base 309 distally against the spring 311, thereby compressing the spring 311. Distal translation of the sliding base 309 causes distal translation of the second inner tube 118 (and second loop 114) relative to the first inner tube 116 (and first loop 112), such that the rounded end portions 112e and 114e of the first loop 112 and second loop 114, respectively, are brought closer together. When the clamshell arms 110a, 110b are fully compressed, the rounded end portions 112e and 114e may contact each other to grasp a flap of a membrane.

From the closed configuration, the user may release the clamshell arms 110a, 110b (i.e., release the pressure urging the clamshell arms 110a, 110b together, thereby actuating the clamshell arms 110a, 110b in second direction 390), which allows the spring 311 to decompress and act upon the sliding base 309 to return the sliding base 309, the piston 307, and the clamshell arms 110a, 110b back to their initial, open positions. This movement also causes the second inner tube 118 and second loop 114 attached thereto to slide back proximally, such that the rounded end portions 112e and 114e of the first loop 112 and the second loop 114, respectively, are moved further apart. Accordingly, in such embodiments, transitioning from the open position to the closed position moves the second loop 114 towards the first loop 112, and transitioning from the closed position to the open position moves the second loop 114 away from the first loop 112.

FIG. 3C is a partial cross-sectional perspective view illustrating another exemplary mechanism for actuating the loops 112 and 114 of the surgical instrument 100. Because FIG. 3C is shown as a cross-sectional view, some or all of the components shown in FIG. 3C may be replicated across a longitudinal plane for components of the other half of the surgical instrument 100. Further, the illustrated mechanism is only exemplary, and the actual size and relative position of components may vary. For purposes of FIG. 3C, “first actuator” refers to the slider 108 and “second actuator” refers to clamshell arms 110a, 110b.

As shown, the embodiments of FIG. 3C are substantially similar to those of FIG. 3B, and operate in substantially the same manner. That is, the clamshell arms 110a, 110b (clamshell arm 110b is shown in FIG. 3B) are operably coupled to the second inner tube 118 (attached to the second loop 114) to actuate the second loop 114, and the slider 108 is operably coupled to the outer tube 106 disposed over the second inner tube 118 to actuate the outer tube 106.

However, unlike the embodiments of FIG. 3B, the cavity 324 in FIG. 3C is formed in the piston 307, rather than in the sliding base 309. Thus, the pin 317 is fixedly attached to the adapter 313 and extends into the cavity 324 formed in the piston 307. Similarly, the stationary first inner tube 116 extends into the cavity 324 in the piston 307 and fixedly attaches to the pin 317 therein, rather than attaching to the pin 317 within the sliding base 309. In such embodiments, the piston 307 is able to translate distally and proximally without being impeded by the pin 317 as a result of the cavity 324 formed therein. To facilitate extension of the first inner tube 116 to the cavity 324 in the piston 307, the conduit 323 in the sliding base 309 extends axially through an entire length of the sliding base 309, rather than only through a portion thereof. Accordingly, but for the location of the cavity 324 and the pin 317, the embodiments of FIG. 3C can operate in substantially the same manner as those of FIG. 3B.

In the embodiments of FIGS. 3B and 3C, the second inner tube 118 is configured to be translated along the longitudinal plane 120a to move the second loop 114 relative to the first loop 112, while the first inner tube 116 is stationary. In certain other embodiments, however, the first inner tube 116 is configured to be translated along the longitudinal plane 120 to move the first loop 112 relative to the second loop 114, while the second inner tube 118 is stationary. FIG. 3D illustrates an exemplary actuation mechanism for such embodiments.

Turning to FIG. 3D, a partial cross-sectional perspective view of another exemplary mechanism for actuating the loops 112 and 114 of the surgical instrument 100 is shown. Because FIG. 3D is shown as a cross-sectional view, some or all of the components shown in FIG. 3D may be replicated across a longitudinal plane for components of the other half of the surgical instrument 100. Further, the illustrated mechanism is only exemplary, and the actual size and relative position of components may vary. For purposes of FIG. 3D, “first actuator” refers to the slider 108 and “second actuator” refers to clamshell arms 110a, 110b.

As shown, the embodiments of FIG. 3D are substantially similar to those of FIGS. 3B and 3C. However, in FIG. 3D, the clamshell arms 110a, 110b (clamshell arm 110b is shown in FIG. 3B) are operably coupled to the first inner tube 116 (attached to the first loop 112) instead of the second inner tube 118 to actuate the first loop 112, and the slider 108 is operably coupled to the outer tube 106 disposed over the second inner tube 118 to actuate the outer tube 106.

To facilitate coupling of the first inner tube 116 with the clamshell arms 110a, 110b, the second inner tube 118 extends through the conduit 323 of the sliding base 309 and into the cavity 324 to fixedly attach to the pin 317, instead of the first inner tube 116. Meanwhile, the first inner tube 116 extends through the conduit 323 and through an entire axial length of the cavity 324 to fixedly couple to the sliding base 309 at a proximal end of the sliding base 309. Accordingly, in such embodiments, the second inner tube 118 and thus, the second loop 114, are stationary due to the pin 317, while the first inner tube 116 and thus, the first loop 112, are configured to translate upon actuation of the clamshell arms 110a, 110b (and the movement of piston 307 and sliding base 309 as a result of the actuation of the clamshell arms 110a, 110b). In such embodiments, the sliding base 309 is able to translate distally and proximally without being impeded by the pin 317 as a result of the cavity 324 formed therein.

Various alternatives to the illustrated embodiments of FIGS. 3B-3D are also contemplated. For example, in certain embodiments, the first inner tube 116 or the second inner tube 118 are operably coupled to the slider 108 to facilitate translation thereof, while the outer tube 106 is operably coupled to the clamshell arms 110a, 110b. In still other embodiments, other types of actuators are also contemplated.

FIG. 3E is a cross-sectional schematic view illustrating another mechanism for actuating the loops 112 and 114 of the grasping structure 104. FIG. 3E may be representative of the embodiments of the surgical instrument 100 in FIG. 2, wherein the clamshell arms 110a, 110b are replaced with the second slider 200. In the illustrated embodiments, the sliders 108, 200 both slide within a common slot 202 defined by the handle 102. Accordingly, one slider 108 may control actuation of the outer tube 106, and the other slider 200 may control actuation of the first inner tube 116, or vice versa. Similar to the above, the illustrated mechanism is only exemplary and shows an example relative movement of components. However, the actual size and relative position of components may vary. For purposes of FIG. 3E, “first actuator” and “second actuator” refer to the slider 108 and the slider 200, respectively.

In the illustrated embodiments, the second inner tube 118 is fixed relative to the handle 102. The outer tube 106 is slidable relative to the handle 102 and has the slider 108 operably coupled thereto, which is also slidable relative to the handle 102. The outer tube 106 defines a slot 302 and the second inner tube 118 defines a slot 304. The second slider 200 is operably coupled to the first inner tube 116 and is slidable within the slots 302, 304, and 202. Similarly, the slider 108 is slidable within the slot 202.

Various alternatives to the illustrated configuration are possible. For example, the first inner tube 116 may be fixed relative to the handle 102 and the second slider 200 may be fastened to the second inner tube 118, which may be slidable relative to the handle 102.

In use, the first slider 108 (e.g., first actuator) may be moved in a first direction 360 to move the outer tube 106 outwardly from the handle 102 and over the first loop 112 and the second loop 114. The first slider 108 may be moved in a second direction 370 opposite the first direction to move the outer tube 106 inwardly thereby extending the first loop 112 and the second loop 114 from the distal end of the outer tube 106. The second slider 200 may be moved in the first direction 360 to move the first loop 112 away from the second loop 114. The second slider 200 may be moved in the second direction 370 to move the first loop 112 toward the second loop 114 in order to grasp a flap of a membrane.

FIG. 3F is a cross-sectional isometric view of an alternative loop connection 301-2 configuration of the surgical instrument shown in FIG. 1A, according to certain embodiments. The loop connection configuration 301-2 may replace the loop connection configuration 301-1 of the surgical instrument 100 shown in 3A.

As opposed to the loop connection configuration 301-1, in loop connection configuration 301-2, ends 312a, 312b of a first loop (e.g., first loop 112 (FIG. 1A)) are attached to a first half rod 316a, and ends 314a, 314b of a second loop (e.g., second loop 114 (FIG. 1A)) are attached to a second half rod 316b. Similar to the first inner tube 116 and the second inner tube 118 above, the first half rod 316a and/or the second half rod 316b are configured to slide, or translate, along or relative to the other to facilitate actuation of the first and second loops coupled thereto.

In certain embodiments, the first half rod 316a and/or the second half rod 316b are made of nitinol, stainless steel, spring steel, rigid polymer, or other material. In some embodiments, the first half rod 316a and the second half rod 316b are disposed within an outer tube (e.g., outer tube 106 (FIG. 1A)).

The first half rod 316a includes a center portion 318a configured to align with a cutout portion 318b of the second half rod 316b, which receives the center portion 318a. In certain embodiments, the first half rod 316a and the second half rod 316b act like sliding rails to guide one another linearly. That is, the first half rod 316a is slidably disposed against the second half rod 316b via the center portion 318a and the cutout portion 318b. Because the first half rod 316a is slidably disposed against the second half rod 316b, the first half rod 316a and/or the second half rod 316b may be connected to one or more manual control structures (e.g., actuation mechanisms) configured to move the first half rod 316a relative to the second half rod 316b, or vice versa. For example, the first half rod 316a traverses along the second half rod 316b via an actuator (e.g., slider 108 or clamshell arms 110a, 110b (FIG. 1A)), which may be coupled to the first half rod 316a. By moving the first half rod 316a along the second half rod 316b, the first loop can be moved to engage with the second loop.

The ends 312a, 312b are disposed in the center portion 318a of the first half rod 316a, and the ends 314a, 314b are disposed on an outer surface of the second half rod 316b. The ends 312a, 312b and the ends 314a, 314b may both be disposed along a horizontal axis 320a, such that the ends 312a, 312b and the ends 314a, 314b are also symmetrically disposed across a vertical axis 320b. The ends 312a, 312b and the ends 314a, 314b may be attached to the first half rod 316a and the second half rod 316b, respectively, by welding, an adhesive (e.g., glue), a pin, a screw, or other similar attaching feature.

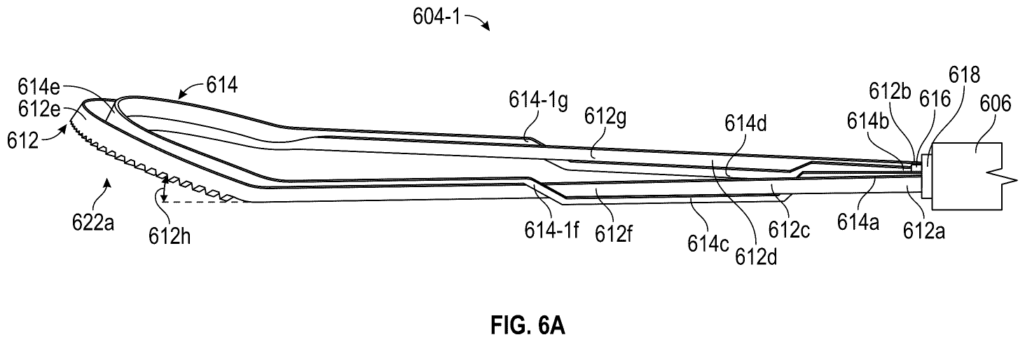

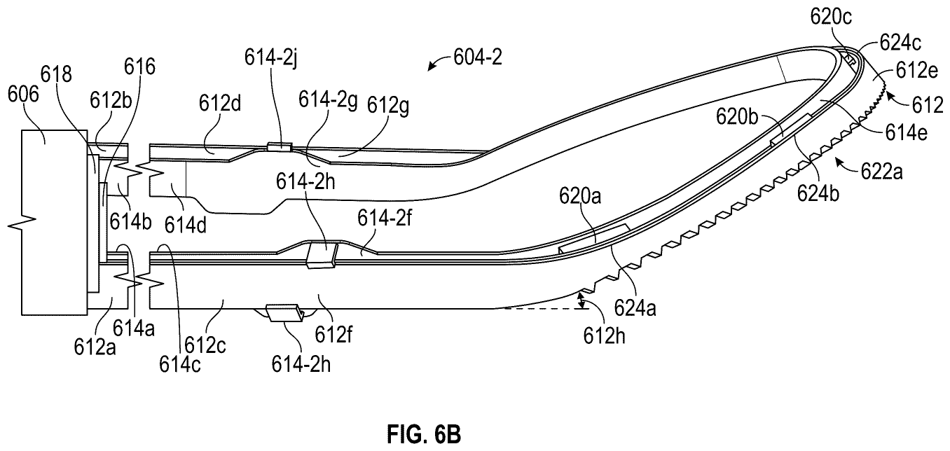

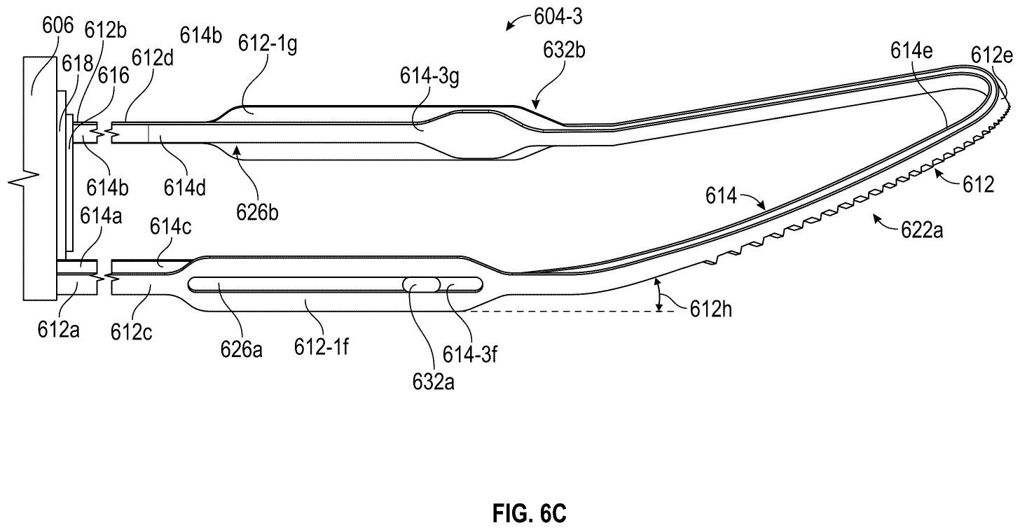

In some embodiments, the ends 312a, 312b and the ends 314a, 314b are switched to match the configuration of the first loop 612 and the second loop 614 as described with reference to FIGS. 6A-6C. That is, the ends 314a, 314b would be attached to the first half rod 316a, and the ends 312a, 312b would be attached to the second half rod 316b.

FIGS. 4A-4B are isometric views showing the grasping structure of FIG. 1A in various configurations, according to certain embodiments.

FIG. 4A is an isometric view showing the grasping structure 104 of FIG. 1A in an open configuration, according to certain embodiments. In FIG. 4A, the guiding portions 112f, 112g of the first loop 112 are disposed through the slots 114f, 114g of the second loop 114, respectively. The second loop 114 is slidable along the first loop 112 via the slots 114f, 114g for at least part of the range of motion of the second loop 114, e.g., for some range of motion ending with the rounded end portion 114e pressed against the rounded end portion 112e. In other words, the second loop 114 can engage with the first loop 112 to grasp an ILM flap by traversing along the first loop 112 via the slots 114f, 114g.

The engagement of the slots 114f, 114g with the guiding portions 112f, 112g may function to maintain alignment of the loops with one another during use. For example, the engagement may prevent the second loop 114 from being displaced above or below the first loop 112 and failing to engage the ILM flap. However, using flexibility of the first loop 112 and second loop 114, both loops may be pressed against the membrane ensuring that the second loop 114 will engage the flap when moved toward the first loop 112. Accordingly, the slots 114f, 114g and the guiding portions 112f, 112g may be omitted in some embodiments.

In preparation for raising a flap, the first loop 112 and the second loop 114 may be positioned in the illustrated open configuration with a gap 150 between the rounded end portion 112e and the rounded end portion 114e that is many times greater than the thickness of the membrane, e.g., at least 10, 100, or 1000 times the thickness of the membrane. Separation of the first loop 112 and the second loop 114 in the open configuration, e.g., via the gap 150, can improve a surgeon's visibility of the first loop 112′s engagement with the membrane and limits interference by the second loop 114 when creating a flap in the membrane.

FIG. 4B is an isometric view showing the grasping structure 104 of FIG. 1A in a closed configuration, according to certain embodiments. The surgeon may translate or manipulate the actuator (e.g., compress the clamshell arms 110a, 110b) in a first direction in order to urge the second loop 114 toward the first loop 112 to achieve the illustrated closed configuration. As is apparent, the rounded end portion 114e will nest within the rounded end portion 112e, thereby firmly grasping a flap raised by the rounded end portion 112e.

While in the closed configuration, the grasping structure 104 may be used to grasp the flap and peel the membrane. The engagement of the first loop 112 and the second loop 114 in the closed configuration improves the surgeon's grip of the flap and reduces the risk for retinal shredding, as compared to conventional methods.

To transition from the closed configuration shown in FIG. 4B to the open configuration shown in FIG. 4A, the surgeon may translate the actuator (e.g., decompress the clamshell arms 110a, 110b) in a second direction (opposite the first direction). Translating the actuator in the second direction moves the second loop 114 away from the first loop 112.

In certain embodiments, the outer tube 106 may be extended partially or completely over the first loop 112 and second loop 114 during use of the surgical instrument 100. The stiffness of the first loop 112 and second loop 114 may be increased by extending the outer tube 106 and reducing the portions of the first loop 112 and second loop 114 that are positioned outwardly from the outer tube 106. Likewise, where more flexibility is desired, the outer tube 106 may be withdrawn to the point that more, potentially the entirety, of the first loop 112 and second loop 114 are exposed.

As noted above, in preparation for insertion of the outer tube 106 through a cannula, the outer tube 106 may be extended until either (a) the first loop 112 and second loop 114 are located completely within the outer tube 106 or (b) the parts of the first loop 112 and second loop 114 extending outwardly form the outer tube 106 are small enough to fit through the cannula (e.g., equal to or smaller than the outer diameter of the outer tube 106).

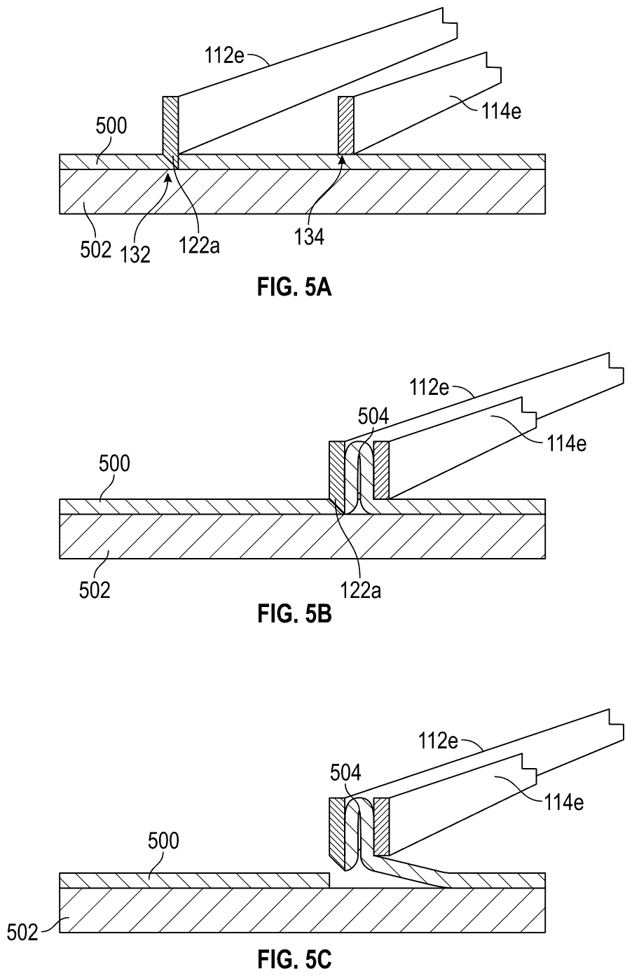

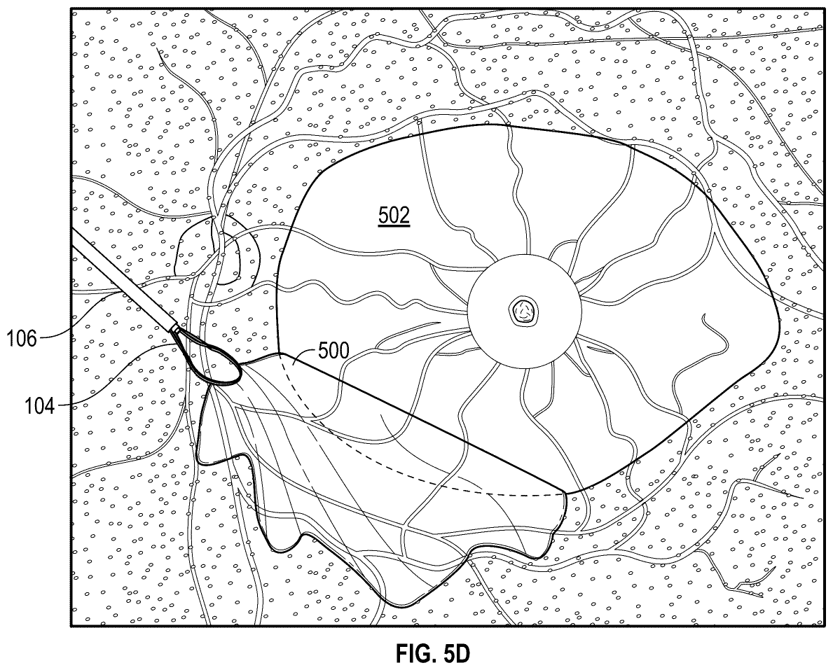

FIGS. 5A-5D show the peeling of an ILM using the grasping structure 104 of FIG. 1A, according to certain embodiments.

Referring now to FIG. 5A, during use, lower surfaces 132-1, 132-2 of the rounded end portion 112e, 114e are pressed against the membrane 500 (e.g., ILM or ERM) positioned over the retina 502. As shown, the first protruding features 122a may at least partially penetrate the ILM. The extent of the first protruding features 122a below the lower surface of the rounded end portion 112e, 114e may be less than the thickness of the ILM, such as less than 2 to 20 microns. For example, the first protruding features 122a may have a length outward from the lower surface of between 0.8 and 8 microns. Referring to FIG. 5B, a flap 504 may be raised by drawing the rounded end portion 112e across the membrane 500 and the rounded end portion 114e may be urged toward the rounded end portion 112e in order to grasp the flap 504 firmly. Referring to FIG. 5C, the surgeon may then lift the surgical instrument 100 in order to tear the membrane 500. Referring to FIG. 5D, the surgeon may move the grasping structure 104 in a circular motion to peel a portion of the membrane 500 away from the retina 502.

Various alternatives to the illustrated method of use of the surgical instrument 100 are possible. For example, the second loop 114 may be fixed relative to the handle 102 as described above and the first loop 112 may be actuated. The first loop 112 may therefore be actuated to move the rounded end portion 112e toward the rounded end portion 114e and thereby both raise the flap 504 and grasp the flap 504 between the rounded end portion 112e and the rounded end portion 114e in a single motion. In other methods of use, the rounded end portion 112e is drawn across the membrane 500 in the direction of the rounded end portion 114e to raise the flap 504 without decreasing the distance between the rounded end portion 112e and the rounded end portion 114e. The rounded end portion 112e is then drawn toward the rounded end portion 114e using the second actuator, which will further raise the flap 504 and grasp the flap 504 between the rounded end portion 112e and the rounded end portion 114e.

Further, although the rounded end 114e of the second loop 114 is shown as contacting the membrane 500 in FIG. 5A, in some embodiments, only the rounded end portion 112e of the first loop 112 contacts the membrane 500 when creating the flap 504. As such, the rounded end portion 112e may be pulled along the surface of the membrane 500 to tear the membrane 500 and create the flap 504. Once the flap 504 is created, the rounded end portion 114e engages with the rounded end portion 112e to grasp the flap 504 as shown in FIG. 5C. Using the flap 504, the membrane 500 can then be peeled as shown in FIG. 5D.

FIG. 6A-6C are isometric views of alternative grasping structures, according to certain embodiments.

FIG. 6A is an isometric view of an alternative grasping structure 604-1, according to certain embodiments. The grasping structure 604-1 may replace the grasping structure 104 of the surgical instrument 100 and may be configured to create and grasp a flap of membrane similar to as described with reference to FIGS. 5A-5D. The grasping structure 604-1 includes the outer tube 606, the first loop 612, and the second loop 614 as described with reference to FIG. 1A. That is, the first loop 612 comprises ends 612a, 612b, straight portions 612c, 612d, guiding portions 612f, 612g, a rounded end portion 612e, and a first plurality of protruding features 622a, and the second loop 614 comprises ends 614a, 614b, straight portions 614c, 614d, and a rounded end portion 614e as described with reference to FIG. 1A.

However, as opposed to the grasping structure 104, the second loop 614 of the grasping structure 604-1 comprises wrap-around portions 614-1f, 614-1g. Additionally, the ends 612a, 612b are fastened to the second inner tube 618 and the ends 614a, 614b are disposed within the first inner tube 616. As such, the ends 614a, 614b and the straight portions 614c, 614d, are inside of (or disposed within) the ends 612a, 612b, and the straight portions 612c, 612d. The wrap-around portions 614-1f, 614-1g extend around (e.g., encircle, intertwine with, wind around, etc.) the guiding portions 612f, 612g starting from inside the first loop 612, thus serving as brackets or guides. In other words, starting from a proximal end of the second loop 614, the wrap around portions 614-1f, 614-1g diverge from one another in the longitudinal transverse plane, traverse along outer surfaces of guiding portions 612f, 612g, then return inwards. That is, the wrap around portions 614-1f, 614-1g flare outwardly from the straight portions 614c, 614d, wrap around the guiding portions 612f, 612g, and then slightly converge inwards before transitioning into the rounded end portion 614e. The wrap around portions 614-1f, 614-1g may be described as having an “S-shape,” or other similar crossover morphology.

In certain embodiments, the wrap around portions 614-1f, 614-1g have the same height or a reduced height (e.g., perpendicular to the longitudinal-transverse plane) and/or thickness (e.g., parallel to the longitudinal-transverse plane) relative to one or both of the rounded end portion 612e and the guiding portions 612f, 612g. In some embodiments, the height and thickness of the wrap around portions 614-1f, 614-1g is substantially (e.g., within 10 percent of) the same as that of the guiding portions 612f, 612g and the rounded end portion 612e. In some embodiments, the height of the wrap around portions 614-1f, 614-1g may be between 0.25 and 0.75 times or between 0.4 and 0.6 times the height of the guiding portions 612f, 612g and the rounded end portion 612e.

In some implementations, the guiding portions 612f, 612g further define a bend in the absence of any deforming force such that the rounded end portion 612e defines an angle 612h relative to the longitudinal-transverse plane and is raised above the longitudinal-transverse plane. The angle 612h may further encourage the rounded end portion 612e to rotate when pushed against the membrane rather than possibly puncturing the retina. The rounded end portion 614e may also be angled relative to the longitudinal-transverse plane by the same angle 612h or a different angle. Flexibility of the first loop 612 and second loop 614 enable the first loop 612 and second loop 614 to nest regardless of size when undeformed.

The second loop 614 engages with the first loop 612 to grasp a flap by traversing along the guiding portions 612f, 612g, as guided or steered by the wrap around portions 614-1f, 614-1g wrapped around the first loop 612. Thus, the wrap around portions 614-1f, 614-1g facilitate proper movement of the second loop 614 along the first loop 612. The second loop 614 traverses along the first loop 612 via an actuator (e.g., slider 108 or clamshell arms 110a, 110b (FIG. 1A)), which may be coupled to the first inner tube 616. The actuator is configured to move the first inner tube 616 relative to the second inner tube 618, which therefore moves the second loop 614 relative to the first loop 612.

FIG. 6B is an isometric view of an alternative grasping structure 604-2, according to certain embodiments. The grasping structure 604-2 may replace the grasping structure 104 of the surgical instrument 100 and may be used to create and grasp the flap as described with reference to FIGS. 5A-5D. The grasping structure 604-2 includes the outer tube 606, the first loop 612, and the second loop 614 as described with reference to FIG. 1A. That is, the first loop 612 comprises ends 612a, 612b, straight portions 612c, 612d, guiding portions 612f, 612g, a rounded end portion 612e, and the first plurality of protruding features 622a, and the second loop 614 comprises ends 614a, 614b, straight portions 614c, 614d, and a rounded end portion 614e, where the rounded end portions 612e, 614e define angle 612h relative to the longitudinal-transverse plane as described with reference to FIG. 1A.

However, as opposed to the grasping structure 104, the second loop 614 of the grasping structure 604-1 comprises bracket portions 614-2f, 614-2g and protruding features 620a, 620b, 620c received by receiving features 624a, 624b, 624c of the first loop 612. Additionally, the ends 612a, 612b are fastened to the second inner tube 618 and the ends 614a, 614b are disposed within the first inner tube 616.

The bracket portions 614-2f, 614-2g extend over the guiding portions 612f, 612g from inside the first loop 612, i.e., the bracket portions 614-2f, 614-2g protrude outwards (e.g., laterally) from within the first loop 612. Further, the bracket portions 614-2f, 614-2g each include a pair of protruding features 614-2h, 614-2j, which partially extend over the guiding portions 612f, 612g. As such, the guiding portions 612f, 612g are disposed through the bracket portions 614-2f, 614-2g. The bracket portions 614-2f, 614-2g and the protruding features 614-2h, 614-2j are configured to guide the second loop 614 towards the first loop 612 as the loops are translated relative to each other. Although the bracket portions 614-2f, 614-2g are shown as partially extending across and over the guiding portions 612f, 612g, the bracket portions 614-2f, 614-2g may also completely extend across and around the guiding portions 612f, 612g on one or both sides.

In certain embodiments, the bracket portions 614-2f, 614-2g may have an increased height (e.g., perpendicular to the longitudinal-transverse plane) and/or thickness (e.g., parallel to the longitudinal-transverse plane) relative to one or both of the rounded end portion 612e and the guiding portions 612f, 612g. In some embodiments, the height of the bracket portions 614-2f, 614-2g may be between 1.25 and 1.75 times or between 1.4 and 1.6 times the height of the guiding portions 612f, 612g and the rounded end portion 612e.

The second loop 614 engages with the first loop 612 to grasp a flap by traversing along the guiding portions 612f, 612g as guided by the bracket portions 614-2f, 614-2g extended over the first loop 612. Thus, the bracket portions 614-2f, 614-2g facilitate proper movement of the second loop 614 along the first loop 612. The second loop 614 traverses along the first loop 612 via an actuator (e.g., slider 108 or clamshell arms 110a, 110b (FIG. 1A)), which may be coupled to the first inner tube 616. The actuator is configured to move the first inner tube 616 relative to the second inner tube 618, which therefore moves the second loop 614 relative to the first loop 612.

The protruding features 620a, 620b, 620c protrude outwards (e.g., extend away from) the rounded end 614e of the second loop 614 and are received by receiving features 624a, 624b, 624c of the first loop 612. The protruding features 620a, 620b, 620c are configured to align the rounded end portion 614e with the rounded end portion 612e when the loops 612 and 614 are engaged. The protruding features 620a, 620b, 620c may have a rectangular or block-like shape and may be equidistantly positioned along an outer surface of the rounded end 614e. For example, protruding feature 620b is positioned at the middle of the rounded end portion 614e and protruding features 620a, 620c are positioned at ends of the rounded end portion 614e.

The receiving features 624a, 624b, 624c are indentations in the rounded end portion 612e that correspond to a shape and positioning of the protruding features 620a, 620b, 620c. Therefore, the receiving features 624a, 624b, 624c may also have a rectangular or block-like shape and may be equidistantly positioned along an inner surface of the rounded end portion 612e. For example, receiving feature 624b is positioned at the middle of the rounded end portion 612e and receiving features 624a, 624c are positioned at ends of the rounded end portion 612e. In certain embodiments, the first loop 612 does not include receiving features 624a, 624b, 624c. As such, the protruding features 620 engage with a top surface of the rounded end portions 612e that is opposite of the surface with the first plurality of protruding features 622a.

Although three protruding features 620a, 620b, 620c and three receiving features 624a, 624b, 624c are shown, there may be less than or more than three protruding features and/or receiving features. The protruding features 620a, 620b, 620c and the receiving features 624a, 624b, 624c may also have different corresponding shapes such as, e.g., semi-circular shapes, triangular shapes, or any other shape capable of interlocking. The protruding features 620a, 620b, 620c and the receiving features 624a, 624b, 624c may also be a combination of different shapes.

The protruding features 620a, 620b, 620c engage with the receiving features 624a, 624b, 624c to help guide and/or align the second loop 614 with the first loop 612 when transitioning to the closed configuration and remain aligned once in the closed configuration. Additionally, engagement of the protruding features 620a, 620b, 620c with the receiving features 624a, 624b, 624c in the closed configuration may improve the grasp of the flap, which reduces the risk for membrane shredding.

FIG. 6C is an isometric view of an alternative grasping structure 604-3, according to certain embodiments. The grasping structure 604-3 may replace the grasping structure 104 of the surgical instrument 100 and may be used to create and grasp a flap of membrane similar to as described with reference to FIGS. 5A-5D. The grasping structure 604-3 includes the outer tube 606, the first loop 612, and the second loop 614 as described with reference to FIG. 1A. That is, the first loop 612 comprises ends 612a, 612b, straight portions 612c, 612d, a rounded end portion 612e, and the first plurality of protruding features 622a, and the second loop 614 comprises ends 614a, 614b, straight portions 614c, 614d, and a rounded end portion 614e, where the rounded end portions 612e, 614e define angle 612h relative to the longitudinal-transverse plane as described with reference to FIG. 1A.

However, as opposed to the grasping structure 104, the first loop 612 comprises slot portions 612-1f, 612-1g with slots 626a, 626b, and the second loop 614 comprises pin portions 614-3f, 614-3g with a pair of pins 632a, 632b. Additionally, the ends 612a, 612b are fastened to the second inner tube 618 and the ends 614a, 614b are disposed within the first inner tube 616. The pins 632a, 632b extend through (e.g., protrude outwards), and are slidably positioned in the slots 626a, 626b. The slots 626a, 626b may function as flexible guiding portions for the second loop 614. For example, when the second loop 614 is traversed along the first loop 612, the slots 626a, 626b facilitate proper movement of the second loop 614 along the first loop 612.

In certain embodiments, the pin portions 614-3f, 614-3g with may have the same height or a reduced height (e.g., perpendicular to the longitudinal-transverse plane) relative to the slot portions 612-1f, 612-1g. In some embodiments, the height of the pin portions 614-3f, 614-3g is substantially (e.g., within 10 percent of) the same as that of the slot portions 612-1f, 612-1g. In some embodiments, the height of the pin portions 614-3f, 614-3g may be between 0.25 and 0.75 times or between 0.4 and 0.6 times the height of the slot portions 612-1f, 612-1g.

The slots 626a, 626b may define an opening having the same height or an increased height (e.g., perpendicular to the longitudinal-transverse plane) relative to the pins 632a, 632b. In some embodiments, the height of the slots 626a, 626b is substantially (e.g., within 10 percent of) the same as that of the pins 632a, 632b. The opening defined by the slots 626a, 626b may have a width that is long enough to accommodate the second loop 614 in its closed configuration and open configuration. Although the slots 626a, 626b are shown as having a semi-circular shaped opening, the slots 626a, 626b may also have a rectangular or other similar shaped opening.

The second loop 614 engages with the first loop 612 to grasp a flap by traversing along the slot portions 612-1f, 612-1g, as guided or steered by the pins 632a, 632b in the slots 626a, 626b. Thus, the pin portions 614-3f, 614-3g facilitate proper movement of the second loop 614 along the first loop 612. The second loop 614 traverses along the first loop 612 via an actuator (e.g., slider 108 or clamshell arms 110a, 110b (FIG. 1A)), which may be coupled to the first inner tube 616. The actuator is configured to move the first inner tube 616 relative to the second inner tube 618, which therefore moves the second loop 614 relative to the first loop 612.

In FIGS. 6A-6C, various alternatives to the illustrated embodiments of the surgical instrument 100 are also possible. For example, the second loop 614 may be fixed relative to the handle 102 as described above, and the first loop 612 may be actuated. The first loop 612 may therefore be actuated to move the rounded end portion 612e toward the rounded end portion 614e and thereby both raise a flap and grasp the flap between the rounded end portion 612e and the rounded end portion 614e. Further, the first loop 612 and/or the second loop 614 may include more than one plurality of protruding features, for example, as described with reference to FIGS. 1C-1D. Yet, still other arrangements are also contemplated.

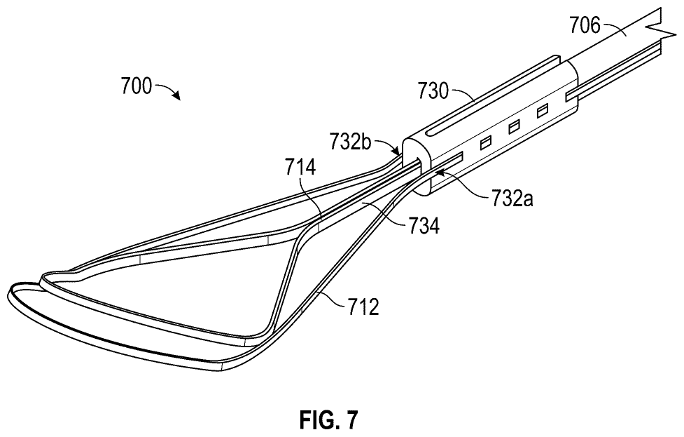

FIG. 7 is an isometric view of another alternative loop connection configuration 700, according to certain embodiments. The loop connection configuration 700 may replace the loop connection configuration 301-1 of the surgical instrument 100 shown in FIG. 3A. The loop connection configuration 700 includes an outer tube 706, a first loop 712, and a second loop 714 as described with reference to FIG. 1A.

As opposed to the loop connection configuration 301-1, the loop connection configuration 700 includes a tip connector 730 disposed over a distal end of the outer tube 706. In other words, the tip connector 730 is disposed over a portion of the outer tube 706. In certain embodiments, the tip connector 730 is attached to the outer tube via welding, an adhesive (e.g., glue), a pin, a screw, or other similar attachment feature. As such, the tip connector 730 may be stationary relative to the outer tube 706. The tip connector 730 may be made of nitinol, stainless steel, spring steel, rigid polymer, or other material.

The first loop 712 is attached to the tip connector 730 at connection points 732a, 732b and may be stationary relative to the second loop 714. The second loop 714 is slidably disposed through a center opening 734 of the tip connector 730 and in certain embodiments, as shown in FIG. 7, is connected to an inner rod slidably disposed within the outer tube 706. The inner rod connected to the second loop 714 may be connected to one or more manual control structures (e.g., actuation mechanisms) configured to move the inner tube and the second loop 714 relative to the first loop 712. For example, the second loop 714 may be moved via an actuator (e.g., slider 108 or clamshell arms 110a, 110b (FIG. 1A)), which may be coupled to the inner rod. As such, the second loop 714 can move relative to the first loop 712 to engage with the first loop 712 by transitioning between open and closed configurations.

The loop connection configuration 700 allows the loops 712 and 714 to be easily serviceable. For example, if the first loop 712 and/or the second loop 714 is damaged, bent, or otherwise deformed, the tip connector 730 can be removed, and the loop(s) can be fixed or replaced. Because the tip connector 730, the first loop 712, and/or the second loop 714 (and inner rod) can be replaced, the surgical instrument may also be reused while still maintaining adequate sterility.

The present disclosure may be embodied in other specific forms without departing from its spirit or essential characteristics. The described embodiments are to be considered in all respects only as illustrative and not restrictive. The scope of the present disclosure is, therefore, indicated by the appended Claims rather than by this Detailed Description. All changes which come within the meaning and range of equivalency of the Claims are to be embraced within their scope.

Reference throughout this specification to features, advantages, or similar language does not imply that all the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the present disclosure.

Reference throughout this specification to “one embodiment”, “an embodiment”, or similar language means that a particular feature, structure, or characteristic described in connection with the indicated embodiment is included in at least one embodiment of the present disclosure. Thus, the phrases “in one embodiment”, “in an embodiment”, and similar language throughout this specification may, but do not necessarily, all refer to the same embodiment.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

### Example Embodiments

Embodiment 1: A ophthalmic surgical instrument, the ophthalmic surgical instrument comprising: a handle; an actuator mounted to the handle; a first loop extending outwardly from the handle and configured to create an internal limiting membrane (ILM) flap in an eye; and a second loop extending outwardly from the handle and positioned within the first loop, the second loop configured to grasp the ILM flap, wherein the actuator is configured to move the second loop to engage the first loop to grasp the ILM flap.
Embodiment 2: The ophthalmic surgical instrument of Embodiment 1, further comprising: an outer tube connected to the handle; a tip connector connected to the outer tube and the first loop, wherein the second loop is disposed through the tip connector and within the outer tube.
Embodiment 3: The ophthalmic surgical instrument of Embodiment 1, wherein the first loop and the second loop each comprise a rounded end portion that defines an angle relative a longitudinal-transverse plane of the handle.
Embodiment 4: The ophthalmic surgical instrument of Embodiment 3, wherein the angle is configured to allow the rounded end portion to rotate when pushed against a membrane of the eye.
Embodiment 5: The ophthalmic surgical instrument of Embodiment 1, wherein second loop comprises: a pair of slots, wherein the first loop is disposed through the pair of slots, and wherein the second loop engages with the first loop to grasp the ILM flap by traversing along the first loop via the pair of slots.
Embodiment 6: The ophthalmic surgical instrument of Embodiment 1, wherein the second loop comprises: one or more portions that wrap around the first loop, wherein the second loop engages with the first loop to grasp the ILM flap by traversing along the first loop as guided by the one or more portions wrapped around the first loop.
Embodiment 7: The ophthalmic surgical instrument of Embodiment 1, wherein the second loop comprises: a pair of brackets that extend over the first loop, wherein the second loop engages with the first loop to grasp the ILM flap by traversing along the first loop as guided by the pair of brackets extending over the first loop.
Embodiment 8: The ophthalmic surgical instrument of Embodiment 1, wherein: the first loop comprises a pair of slots; and the second loop comprises a pair of pins, wherein the second loop engages with the first loop to grasp the ILM flap by traversing along the pair of slots of the first loop as guided by the pair of pins.
Embodiment 9: The ophthalmic surgical instrument of Embodiment 1, wherein: the first loop comprises one or more receiving features; and the second loop comprises one or more protruding features, wherein the one or more receiving features are configured to receive the one or more protruding features.
Embodiment 10: The ophthalmic surgical instrument of Embodiment 1, wherein the actuator is configured to move the second loop toward the first loop responsive to movement of the actuator in a first direction.
Embodiment 11: The ophthalmic surgical instrument of Embodiment 10, herein the actuator is configured to move the second loop away from the first loop responsive to movement of the actuator in a second direction opposite the first direction.

What is claimed is:

1. An ophthalmic surgical instrument for peeling a retinal membrane, comprising:
a handle;
an actuator mounted to the handle;
a first loop extending outwardly from the handle, the first loop comprising:
a first plurality of protruding features extending from a surface of the first loop and configured to grip an internal limiting membrane (ILM) flap in an eye; and
a second loop extending outwardly from the handle and positioned within the first loop and configured to grasp the ILM flap,
wherein the actuator is configured to move the second loop to engage the first loop to grasp the ILM flap.

2. The ophthalmic surgical instrument of claim 1, wherein the second loop comprises:
a pair of slots,
wherein the first loop is disposed through the pair of slots, and
wherein the second loop engages with the first loop to grasp the ILM flap by traversing along the first loop via the pair of slots.

3. The ophthalmic surgical instrument of claim 1, wherein the second loop comprises:
one or more portions that wrap around the first loop,
wherein the second loop engages with the first loop to grasp the ILM flap by traversing along the first loop as guided by the one or more portions wrapped around the first loop.

4. The ophthalmic surgical instrument of claim 1, wherein the second loop comprises:
a pair of brackets that extend over the first loop,
wherein the second loop engages with the first loop to grasp the ILM flap by traversing along the first loop as guided by the pair of brackets extending over the first loop.

5. The ophthalmic surgical instrument of claim 1, wherein:
the first loop comprises a pair of slots; and
the second loop comprises a pair of pins,
wherein the second loop engages with the first loop to grasp the ILM flap by traversing along the pair of slots of the first loop as guided by the pair of pins.

6. The ophthalmic surgical instrument of claim 1, wherein:
the first loop comprises one or more receiving features; and
the second loop comprises one or more protruding features,
wherein the one or more receiving features are configured to receive the one or more protruding features.

7. The ophthalmic surgical instrument of claim 1, further comprising:
a first tube connected to the first loop; and
a second tube connected to the second loop,
wherein the first tube is slidably disposed within the second tube.

8. The ophthalmic surgical instrument of claim 1, further comprising:
a first half rod connected to the first loop; and
a second half rod connected to the second loop,
wherein the second half rod is slidably disposed against the first half rod.

9. The ophthalmic surgical instrument of claim 1, wherein the first plurality of protruding features protrude transversely from a rounded end portion of the first loop.

10. The ophthalmic surgical instrument of claim 1, wherein the first loop comprises:
a pair of sides; and
a rounded end portion connecting the pair of sides, wherein:
the first plurality of protruding features protrude from a first side of the rounded end portion, and
a second plurality of protruding features protrude from a second side of the rounded end portion.

11. The ophthalmic surgical instrument of claim 1, the second loop further comprising:
a second plurality of protruding features configured to grip the ILM flap in the eye.

12. The ophthalmic surgical instrument of claim 1, wherein the actuator is configured to move the second loop toward the first loop responsive to movement of the actuator in a first direction.

13. The ophthalmic surgical instrument of claim 12, wherein the actuator is configured to move the second loop away from the first loop responsive to movement of the actuator in a second direction opposite the first direction.

14. An ophthalmic surgical instrument, the ophthalmic surgical instrument comprising:
a handle;
an actuator mounted to the handle;
a first loop extending outwardly from the handle and configured to create an internal limiting membrane (ILM) flap in an eye; and
a second loop extending outwardly from the handle and positioned within the first loop, the second loop comprising:
a pair of slots,
wherein the actuator is configured to move the second loop to engage the first loop to grasp the ILM flap by traversing along the first loop as guided by the pair of slots.

15. An ophthalmic surgical instrument, the ophthalmic surgical instrument comprising:
a handle;
an actuator mounted to the handle;
a first loop extending outwardly from the handle and configured to create an internal limiting membrane (ILM) flap in an eye; and
a second loop extending outwardly from the handle and positioned within the first loop, the second loop configured to grasp the ILM flap,
wherein the actuator is configured to move the second loop to engage the first loop to grasp the ILM flap;
wherein the first loop and the second loop each comprise a rounded end portion that defines an angle relative to a longitudinal-transverse plane of the handle.
